(19) United States
(12) Patent Application Publication
(10) Pub. No.: US 2005/0075628 A1
(43) Pub. Date: Apr. 7, 2005

(54) Variable intensity wide-angle illuminator

(76) Inventors: Karl Cazzini, Framingham, MA (US); David E. Booth, Wyomissing Hills, PA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/697,422

(22) Filed: Oct. 30, 2003

Related U.S. Application Data
(60) Provisional application No. 60/508,153, filed on Oct. 2, 2003.

(51) Int. Cl. A61B 18/18

(52) U.S. Cl. 606/4

(57) ABSTRACT

A variable-intensity, wide-angle illuminator is disclosed, one embodiment comprising: a light source for providing a light beam; an optical cable, optically coupled to the light source for receiving and transmitting the light beam; a handpiece, operably coupled to the optical cable to receive the light beam; an optical fiber, operably coupled to the handpiece, wherein the optical fiber is optically coupled to the optical cable to receive and transmit the light beam; an optical element, optically coupled to a distal end of the optical fiber, for receiving the light beam and scattering the light beam to illuminate a surgical field, wherein the optical element comprises: a polymer matrix; and a plurality of microbubbles displaced within the polymer matrix; and a cannula, operably coupled to the handpiece, for housing and directing the optical fiber and the optical element. The optical element can be a small-gauge, diffusive optical element having circular or semi-ellipsoidal incident surfaces. For example, the optical element can be a 19, 20 or 25 gauge optical element. Further, the optical element, the cannula and the handpiece can be fabricated from biocompatible materials. The optical cable can comprise a first optical connector operably coupled to the light source and a second optical connector operably coupled to the handpiece (to optically couple the optical cable to the optical fiber housed within the handpiece and cannula).

### TECHNICAL FIELD OF THE INVENTION

This application claims priority from U.S. Ser. No. 60/508,153 filed Oct. 2, 2003.

The present invention relates generally to surgical instrumentation. In particular, the present invention relates to surgical instruments for illuminating an area during eye surgery. Even more particularly, the present invention relates to a variable intensity, small gauge, wide-angle illuminator for illumination of a surgical field.

### BACKGROUND OF THE INVENTION

In ophthalmic surgery, and in particular in vitreo-retinal surgery, it is desirable to use a wide-angle surgical microscope system to view as large a portion of the retina as possible. Wide-angle objective lenses for such microscopic systems exist, but they require a wider illumination field than that provided by the cone of illumination of a typical fiber-optic probe. As a result, various technologies have been developed to increase the beam spreading of the relatively incoherent light provided by a fiber-optic illuminator. These known wide-angle illuminators can thus illuminate a larger portion of the retina as required by current wide-angle surgical microscope systems. Currently existing wide-angle illuminators, however, display several disadvantages.

One disadvantage exhibited by some prior art wide-angle illuminators for ophthalmic surgery is a matching of the light refracting index of the vitreous eye fluid to that of the light refracting surface of the lens of the illuminator that comes in contact with the vitreous eye fluid. Contact of the vitreous eye fluid with the light refracting surface of the light spreading lens of such prior art systems results in sub-optimal light refraction due to index switching caused by the vitreous eye fluid. U.S. Pat. No. 5,624,438, entitled “Retinal Wide-Angle Illuminator For Eye Surgery,” and issued to R. Scott Turner, provides a system for overcoming the effect of refractive index matching through the use of a high refractive-index step, mediated by the presence of an air-gap. The air-gap is presented between the distal end of an optical fiber and the light refracting surface of the illuminator lens. The light emanating from the optical wave guide (i.e., the optical fiber) will therefore undergo angular dispersion without any index switching that might be caused by contact with the vitreous eye fluid before it passes through the light refracting surface of the illuminator lens.

Another disadvantage of currently available wide-angle illuminators is glare. Glare results when the source of the illumination is small and bright, and the user (e.g., an ophthalmic surgeon) has a direct line of sight to the small bright illumination source. Glare is unwanted stray radiation that provides no useful illumination, and either distracts an observer or obscures an object under observation. Glare can be corrected for in current wide-angle illuminators, but typically only by reducing the total illumination light flux, which reduces the amount of light available for observation by the surgeon. For example, the “bullet probe” manufactured by Alcon Laboratories, Inc., of Fort Worth, Tex., achieves wide-angle illumination by using a bullet-shaped fiber having a surface diffusive finish to scatter light emanating from the distal end of an optical fiber. To reduce glare, the bullet probe can use a geometric shield, which reduces the illumination angle by reducing the overall available light flux.

A further disadvantage of typical prior art wide-angle illuminators is that they do not provide for varying the illumination angle and/or the intensity of the light source to adjust illumination for different conditions within the surgical field. Further still, prior art wide-angle surgical illuminators are expensive to produce, a cost which is passed along to the surgeon and ultimately to the patient. As a result, prior art illuminators are typically not disposable and will require periodic maintenance and sterilization between surgical procedures.

Therefore, a need exists for a variable-intensity, wide-angle illuminator that can reduce or eliminate the problems of refractive-index matching, glare, adjustable illumination properties, cost, efficiency and other problems associated with prior art wide-angle illuminators.

### BRIEF SUMMARY OF THE INVENTION

The embodiments of the variable-intensity, wide-angle illuminator for illuminating a surgical field of the present invention substantially meet these needs and others. One embodiment of the variable-intensity, wide-angle illuminator of this invention is a small-gauge, wide-angle illumination surgical system comprising: a light source for providing a light beam; an optical cable, optically coupled to the light source for receiving and transmitting the light beam; a handpiece, operably coupled to the optical cable to receive the light beam; an optical fiber, operably coupled to the handpiece, wherein the optical fiber is optically coupled to the optical cable to receive and transmit the light beam; an optical element, optically coupled to a distal end of the optical fiber, for receiving the light beam and scattering the light beam to illuminate a surgical field, wherein the optical element comprises a polymer matrix and a plurality of microbubbles displaced within the polymer matrix; and a cannula, operably coupled to the handpiece, for housing and directing the optical fiber and the optical element.

The optical element can be a small-gauge, diffusive optical element having circular or semi-ellipsoidal incident surfaces. For example, the optical element can be a 19, 20 or 25 gauge optical element. Further, the optical element, the cannula and the handpiece can be fabricated from biocompatible materials. The optical cable can comprise a first optical connector operably coupled to the light source and a second optical connector operably coupled to the handpiece (to optically couple the optical cable to the optical fiber housed within the handpiece and cannula). These connectors can be SMA optical fiber connectors. The optical element, optical fiber and optical cable (i.e., the optical fibers within the optical cable) should be of a compatible gauge so as to transmit the light beam from the light source to the surgical field. For example, all three elements could be of equal gauge.

To enable some of the advantages of the embodiments of this invention, the optical fiber can be operably coupled to the handpiece to enable linear displacement of the optical fiber and the optical element within the cannula. The handpiece can include a means, such as a push/pull mechanism, for adjusting the linear displacement of the optical fiber and the optical element. Other adjusting means as known to those in the art can also be used. The distal end (end closest to the surgical field) of the optical element can be co-incident with an open aperture of the cannula. Adjusting the linear displacement will thus cause the optical element to exit the open aperture by an amount corresponding to the change in linear displacement (a reverse adjustment can retract the optical element). In this way, the angle of illumination and the amount of illumination provided by the optical element from the light beam to illuminate the surgical field (e.g., the retina of an eye) can be adjusted by the surgeon as needed. Embodiments of this invention can provide a range of illumination angles up to about 180 degrees (e.g., 20 degrees to about 180 degrees).

Other embodiments of the present invention can include a method for wide-angle illumination of a surgical field using a variable-intensity, wide-angle illuminator in accordance with the teachings of this invention, and a surgical handpiece embodiment of the variable-intensity, wide-angle illuminator of the present invention for use in ophthalmic surgery. Embodiments of this invention can be implemented as a handpiece connected to a cannula or other housing including a fiber optic cable terminating in a diffusive optical element in accordance with the teachings of this invention. Further, embodiments of this invention can be incorporated within a surgical machine or system for use in ophthalmic or other surgery. Other uses for a variable-intensity, wide-angle illuminator designed in accordance with the teachings of this invention will be known to those familiar with the art.

### BRIEF DESCRIPTION OF THE SEVERAL VIEWS OF THE DRAWINGS

A more complete understanding of the present invention and the advantages thereof may be acquired by referring to the following description, taken in conjunction with the accompanying drawings, in which like reference numbers indicate like features and wherein:

FIG. 1 is a simplified diagram of one embodiment of a system for variable, wide-angle illumination in accordance with the teachings of this invention;

FIG. 2 is a more detailed diagram of a stem housing an embodiment of a diffusive element for wide-angle illumination in accordance with the teachings of this invention;

FIG. 3 is a diagram illustrating the use of an embodiment of a wide-angle illuminator of the present invention for ophthalmic surgery; and

FIG. 4 is a diagram illustrating an embodiment of an adjusting means 40 in accordance with the present invention.

### DETAILED DESCRIPTION OF THE INVENTION

Preferred embodiments of the present invention are illustrated in the FIGURES, like numerals being used to refer to like and corresponding parts of the various drawings.

The various embodiments of the present invention provide for a small gauge (e.g., 19, 20, or 25 gauge) optical fiber based endo-illuminator device for use in surgical procedures, such as in vitreo-retinal/posterior segment surgery. Embodiments of this invention can comprise a handpiece, such as the Alcon-Grieshaber Revolution-DSP™ handpiece sold by Alcon Laboratories, Inc., Fort Worth, Tex., connected to a small gauge cannula (e.g., 19, 20, or 25 gauge). The inner dimension of the cannula can be used to house one, or a plurality of, optical fibers terminating in a diffusive optical element in accordance with the teachings of this invention. Embodiments of the wide-angle illuminator can be configured for use in the general field of ophthalmic surgery. However, it is contemplated and it will be realized by those skilled in the art that the scope of the present invention is not limited to ophthalmology, but may be applied generally to other areas of surgery where wide-angle and/or variable illumination may be required.

An embodiment of the variable-intensity, wide-angle illuminator of this invention can comprise a light diffusive element, stem and handpiece fabricated from biocompatible polymeric materials, such that the invasive portion of the wide-angle illuminator is a disposable surgical item. Unlike the prior art, each embodiment of the variable-intensity, wide-angle illuminator of this invention can provide high optical transmission/high brightness with low optical losses. Embodiments of this invention fabricated from biocompatible polymeric materials can be integrated into a low cost, articulated handpiece mechanism, such that these embodiments can comprise an inexpensive disposable illuminator instrument.

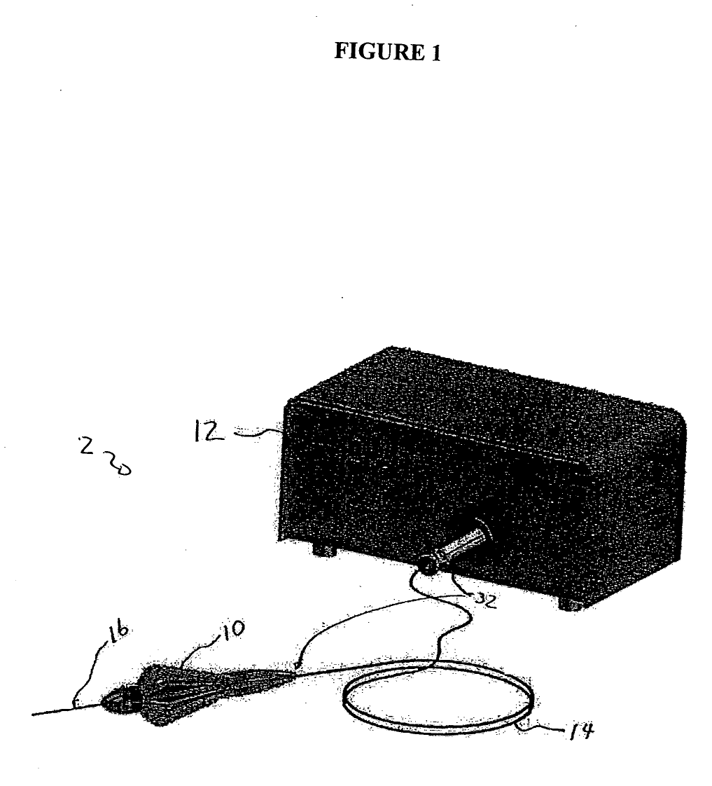

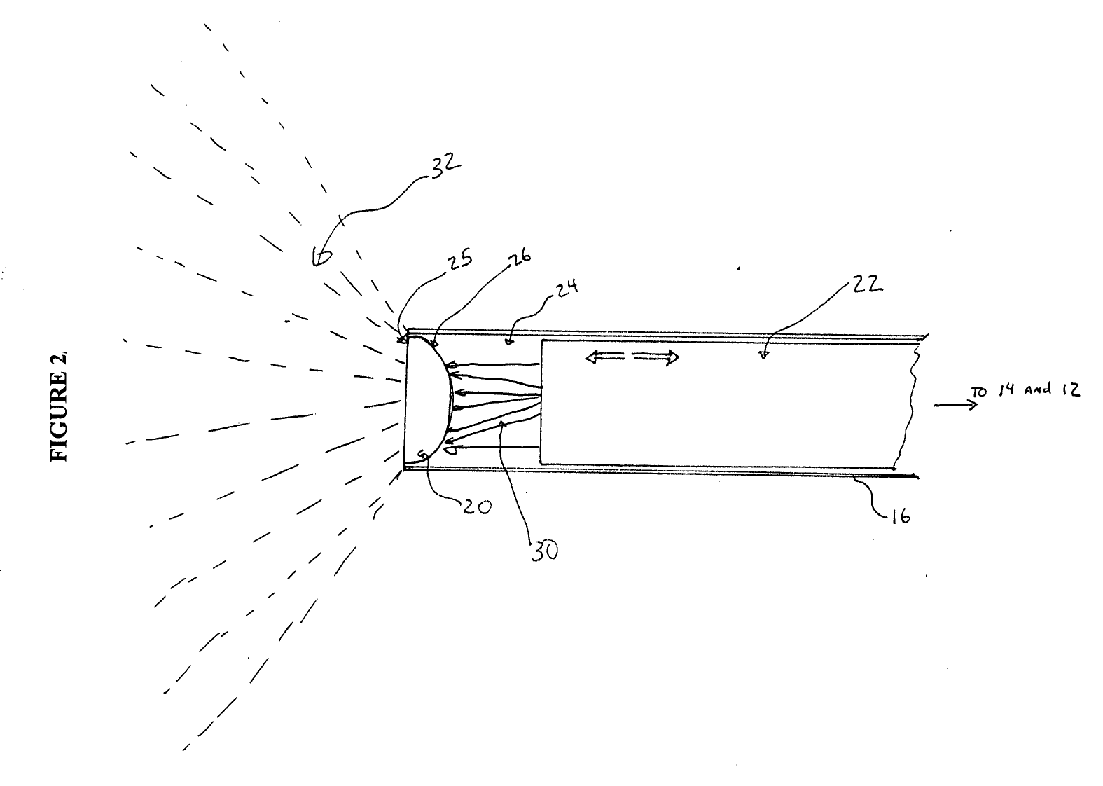

FIG. 1 is a simplified diagram of a handpiece 10 for delivering a beam of incoherent light from a light source 12 through cable 14 to a stem 16. Cable 14 can be any gauge fiber optic cable as known in the art, but is preferably a cable having 19, 20, or 25 gauge fiber. Further, cable 14 can comprise a single optical fiber or a plurality of optical fibers optically coupled to receive and transmit light from light source 12 to stem 16 through handpiece 10. Stem 16 is configured to house a diffusive optical element 20 at the distal end of stem 16, as is more clearly illustrated in FIG. 2. Coupling system 32 can comprise an optical fiber connector at each end of cable 14 to optically couple light source 12 to an optical fiber within handpiece 10, as discussed more fully below.

FIG. 2 is a magnified view of the distal end of stem 16. Stem 16 is shown housing fiber 22 and optical element 20. Optical element 20 is optically coupled to fiber 22, which is itself optically coupled to fiber optic cable 14. In some embodiments, fiber optic cable 14 can extend through the handpiece 10 and is optically coupled directly to optical element 20. For these embodiments, fiber 22 is not used. When implemented within handpiece 10, fiber 22 is of a gauge compatible with the gauge of fiber optic cable 14 such that it can receive and transmit light from fiber optic cable 14. Handpiece 10 can be any surgical handpiece as known in the art, such as the Revolution-DSP™ handpiece sold by Alcon Laboratories, Inc. of Fort Worth, Tex. Light source 12 can be a xenon light source, a halogen light source, or any other light source capable of delivering incoherent light through a fiber optic cable. Stem 16 can be a small gauge cannula, preferably within the range of 18 to 30 gauge, as known to those in the art. Stem 16 can be stainless steel or a suitable biocompatible polymer (e.g., PEEK, polyimide, etc.) as known to those in the art.

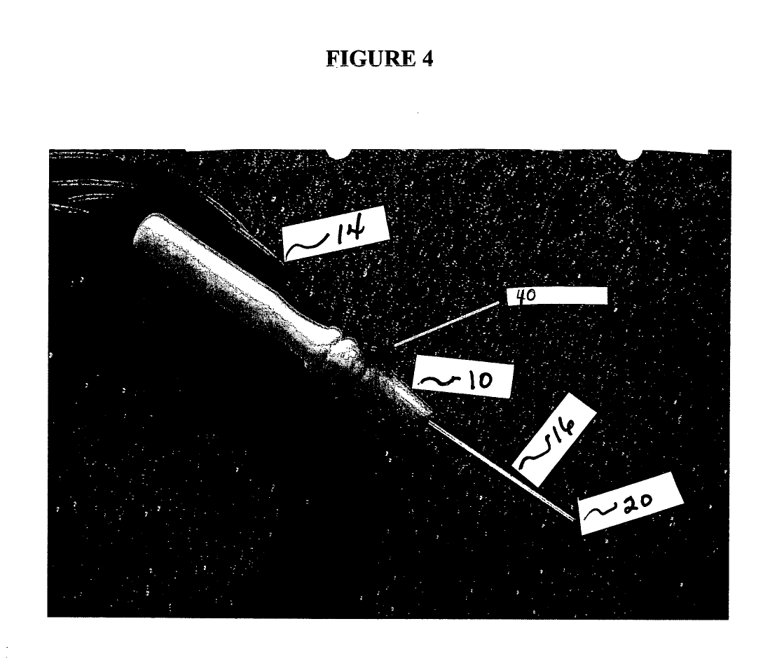

The fiber optic cable 14 or fiber 22 housed within the stem 16 can be operably coupled to the handpiece 10, for example, via an adjusting means 40, as shown in FIG. 4. Adjusting means 40 can comprise for example, a simple push/pull mechanism as known to those in the art. Light source 12 can be optically coupled to handpiece 10 (i.e., to fiber 22) using, for example, standard SMA (Scale Manufacturers Association) optical fiber connectors at the proximal ends of fiber optic cable 14. This allows for the efficient coupling of light from the light source 12 through fiber optic cable 14 to the handpiece 10 and finally emanating from optical element 20 at the distal end of the stem 16. Light source 12 may comprise filters, as known to those skilled in the art, to reduce the damaging thermal effects of absorbed infrared radiation originating at the light source. The light source 12 filter(s) can be used to selectively illuminate a surgical field with different colors of light, such as to excite a surgical dye.

Fiber(s) 22 (and/or 14, depending on the embodiment) is/are terminated by operably and optically coupling to optical element 20. Optical element 20 can be an optical grade polymer diffuser of cylindrical (i.e., circular face) or semi-ellipsoidal cross section. The length of optical element 20 can be about two millimeters. When not in use, optical element 20 can be shielded within stem 16, the distal end of optical element 20 being co-incident with the open aperture at the distal end of stem 16. Activation of the adjusting means 40, by, for example, a gentle and reversible sliding action, can cause optical element 20 to exit (or retract into) the distal end of stem 16 by an amount determined and adjusted by sliding adjusting means 40. The amount of illumination and the solid angle of illumination may be varied according to the amount of optical element 20 which is exposed at the end of stem 16. In this way, a surgeon can adjust the amount of light spread over a surgical field as desired to optimize the viewing field while minimizing glare. The adjusting means 40 of handpiece 10 can be any adjusting means as known to those familiar with the art.

In one embodiment of the variable-intensity, wide-angle illuminator of the present invention, a simple mechanical locking mechanism, as known to those skilled in the art, can permit the illumination angle to be fixed, until released and/or re-adjusted by the user via the adjusting means 40. Light emanating from the distal end of stem 16 will illuminate an area over a solid angle θ, the angle θ being continuously adjustable by a user (e.g., a surgeon) via the adjusting means 40 of handpiece 10. A more detailed explanation of the optical element 20 and its method of fabrication is provided below.

Returning to FIG. 2, a more detailed view of stem 16, including optical fiber 22 and diffusive optical element 20 are shown. As shown more clearly in FIG. 2, optical element 20 comprises a random distribution of microbubbles or microvoids 24 within a polymer matrix 26. Optical element 20 can be physically and optically connected to the distal end of the light carrying fiber 22 housed inside stem 16 (e.g., a small-gauge cannula of about 18 to 30 gauge). Stem 16 is itself operably connected to the handpiece 10, which can be either a re-usable or disposable handpiece 10. Light exiting the distal end of fiber 22 is transmitted into the closely indexed-matched polymer matrix 26, which can comprise a random density distribution of gas-filled, fluid filled or evacuated microbubbles 24.

FIG. 2 illustrates one embodiment of optical element 20 implemented in an endo-illuminator function. The diameter of microbubbles or microvoids 24 is between 1 and 50 microns and preferably between 10 and 25 microns. The microbubbles 24 are distributed with a sufficient distribution density to scatter and transmit the light received from light source 12 in an isotropic manner. The exact scattering properties of the diffusive element 20 are determined by the number-density and the size distribution of the scattering microbubbles 24, as will be apparent to those familiar with the art. Furthermore, but to a lesser extent, the overall shape of the diffusive element 20 will influence the overall light distribution. Diffusive element 20 may be realized through various means, including, but not limited to, the incorporation of microscopic optical scattering and/or refracting centers within an optical polymer matrix 26. This may be achieved in numerous ways known to those skilled in the art. Numerous such methods are described below.

In the first method, diffusive element 20 comprises a cylindrical or ellipsoidal volume of optical grade clear and transparent polymer matrix 26, modified by the introduction of a random array of evacuated, gas-filled or fluid-filled spheroidal microbubbles 24. Suitable materials for the construction of such a polymer matrix 26 may be, by way of example but without limitation, clear optical grade epoxy resins, optically transparent silicone rubbers, uv curable optical adhesives and resins, glasses, optical ceramics, aerogels or other curable optical grade materials as known to those skilled in the art.

Alternatively, microscopic, optically-scattering and refracting hollow microbubbles 24 may be realized in an optically clear polymer matrix 26 (epoxy or UV curable polymer) prior to curing and molding the polymer matrix 26 material. Microbubbles 24 can be made to have a range of diameters, with between about 1 and 50 microns being well suited to act as refracting and scattering centers for visible light transmitted through optical diffusive element 20. In a preferred embodiment, the array of microbubbles 24 has a range of diameters between 10 and 15 microns. Formation of the hollow microbubbles 24 in a polymer matrix 26 may be accomplished in many ways known to those skilled in the art. For example, optical element 20 may be created by incorporation (doping) of thermally expanding polymeric hollow microspheres, such as those manufactured by Emerson and Cuming Incorporated, (e.g., Expancel™), into the uncured polymer matrix 26.

Thermally expanding microspheres, as described above are commonly used for reducing the density of extruded components in the injection molding industry. The doped polymer matrix 26 is molded to the desired final shape of the optical element 20. When treated at elevated temperature, the incorporated thermally expanding microspheres within the polymer matrix 26 enlarge, following a well characterized volume expansion characteristic. Upon completion of curing, polymer matrix 26 comprises a predetermined density multiplicity of essentially gas-filled, low refractive index, scattering centers having a high optical transmission coefficient. Light entering the optical element 20 from fiber 22 is transmitted through the polymer matrix 26. Optical element 20 will scatter received light in an isotropic and highly divergent manner due to the random distribution and the high/low refractive index interfaces associated with the microbubbles 24 and the polymer matrix 26.

Optical diffusive element 20 may also be created by the agitation of a suitably viscous liquid polymer or epoxy resin using an appropriately coupled ultrasound generator. Ultrasonic agitation is well known to those skilled in the art as a means for mixing and cavitating a solution of suitable viscosity. Suitable curing conditions will result in the spatial fixation of a plurality of random microbubbles 24 inside a cured polymer matrix 26. A polymer matrix 26 created in such a manner may be post-machined and polished to the required optical element 20 shape, or molded to the desired geometry prior to initiating the final cure.

Alternately, optical element 20 may be created by passing a polymer solution or uncured epoxy resin, together with a gas, through a microporous membrane under high pressure. This method will induce the random formation of microbubbles 24 within the viscous polymer/epoxy resin 26. Subsequent molding and curing around the distal end of the light carrying optical fiber 22 results in the formation of an optical element 20 of the desired geometry and characteristics.

The fabrication of optical element 20 may be achieved by other methods known to those skilled in the art. Such methods may include, but should not be limited, to the use of aerogels, porous glass, polymer and or silicone foams, vacuum seeding and aeration technologies, all of which can be used to produce a random distribution of hollow microbubbles 24 within a transparent optical medium such as polymer matrix 26.

Furthermore, the optical element 20 may comprise an optically transparent material, within which a random plurality of microvoids or microbubbles is formed using a tightly focused beam of laser radiation, the characteristics of which are generally known to those skilled in the art. A flexible and essentially elastic material, such as silicone rubber, can also be used for the matrix 26 material supporting the glass microbubbles 24. In such an embodiment of this invention the silicone matrix 26 containing the microbubbles 24 may be distorted mechanically within the stem 16, thereby changing its geometry and the distribution of microbubbles 24. This will have the effect of varying the light distribution within the illumination field. The silicone matrix 26 can be distorted mechanically via a mechanism within, for example, handpiece 10.

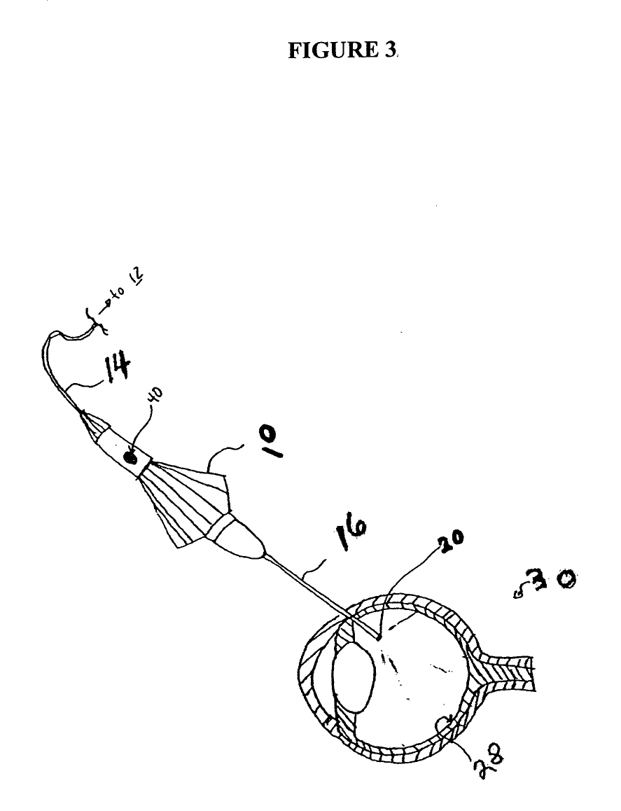

FIG. 3 illustrates the use of one embodiment of the variable-intensity, wide-angle illuminator of this invention in an ophthalmic surgery. In operation, handpiece 10 delivers a beam of spatially and temporally incoherent light having a broad spectral bandwidth through stem 16 (via optical fiber 22) and through optical element 20 to illuminate a retina 28 of an eye 30. The collimated light delivered through handpiece 10 to optical element 20 is generated by light source 12 and delivered to illuminate the retina 28 by means of fiber optic cable 14 and coupling system 32. Optical element 20 spreads the light beam delivered from light source 12 over as large an area of the surgical field as, for example, a microscopic wide-angle objective lens permits a surgeon to see.

An advantage of the optical element 20 and of the embodiments of the variable-intensity, wide-angle illuminator of this invention, is that an operator can continuously vary the intensity and angle of illumination of the light exiting optical element 20 to optimize viewing conditions within the surgical field. The light emanating from optical element 20 can thus be spatially dispersed and controlled as desired by the operator (e.g., surgeon). The embodiments of the variable-intensity, wide-angle illuminator of the present invention are thus operable to adjust the angle and intensity of the light provided by light source 12 to substantially cover the area of the surgical field desired by a surgeon.

The embodiments of the variable-intensity, wide-angle illuminator of this invention provide several advantages over the prior art, such as maximizing light transmission by eliminating the requirement of multiple transmitting, reflecting, or diffracting optical elements, all of which can present sources of further transmission loss between a light source 12 and a target area to be illuminated. Furthermore, the embodiments of this invention have an inherently high light flux capacity and a variable illumination angle, which will permit the operator to tailor the angular illumination requirements for a specific surgical environment. Additionally, a variable illumination angle allows an operator to adjust the intensity of the illumination using both source intensity variations and angle of incidence variations to minimize glare and shadowing in the surgical field. By varying the angle of illumination on a specific portion of the surgical field, an operator, such as a surgeon, can get an improved perception of spatial awareness.

A traditional fiber-optic illuminator with a polished face will produce an included illumination angle that is a function of the numerical aperture (“NA”) of the fiber. NA defines the acceptance angle of entrance of the light from the light source into the fiber optic cable. Commonly, the fiber used for ophthalmic illumination applications has a typical NA of 0.5. This provides a calculated acceptance angle of 60° in vacuo. Wide-angle viewing systems commonly used by ophthalmic surgeons typically have a viewing angle requirement of greater than about 100° in vivo. Thus, conventional fiber optic illuminators cannot provide a lighted field that matches the viewing system angle of visibility. The embodiments of the variable-intensity, wide-angle illuminator of this invention can provide an angle of illumination in excess of about 180° (i.e., a range of illumination angles up to about 180°).

Although the present invention has been described in detail herein with reference to the illustrated embodiments, it should be understood that the description is by way of example only and is not to be construed in a limiting sense. It is to be further understood, therefore, that numerous changes in the details of the embodiments of this invention and additional embodiments of this invention will be apparent to, and may be made by, persons of ordinary skill in the art having reference to this description. It is contemplated that all such changes and additional embodiments are within the spirit and true scope of this invention as claimed below. Thus, while the present invention has been described in particular reference to the general area of ophthalmic surgery, the teachings contained herein apply equally wherever it is desirous to provide wide-angle and variable illumination, and where contact with a transparent fluid might normally interfere with the ability to obtain wide-angle illumination.

1. A small-gauge optical element comprising:
a polymer matrix; and
a plurality of microbubbles displaced within the polymer matrix, wherein the small-gauge optical element is optically coupled to receive a light beam from a light source and to scatter the light beam to illuminate a surgical field.

2. The small-gauge optical element of claim 1, wherein the small-gauge optical element further comprises circular or semi-ellipsoidal incident surfaces.

3. The small-gauge optical element of claim 1, wherein the small-gauge optical element is sized between 18 and 30 gauge and wherein the small-gauge optical element is optically coupled to an optical fiber for receiving the light beam.

4. The small-gauge optical element of claim 3, wherein the optical fiber gauge is equal to the gauge of the small-gauge optical element.

5. The small-gauge optical element of claim 3, wherein the small-gauge optical element and the optical fiber are housed within a cannula.

6. The small-gauge optical element of claim 5, wherein the cannula is operably coupled to a handpiece.

7. The small-gauge optical element of claim 6, wherein the small-gauge optical element, the cannula and the headpiece are fabricated from biocompatible materials.

8. The small-gauge optical element of claim 6, wherein the optical fiber is optically coupled at one end to the small-gauge optical element and at the other end to an optical cable, wherein the optical cable is operably coupled to the light source to transmit the light beam to the optical fiber, and wherein the optical cable comprises a first optical connector operably coupled to the light source and a second optical connector operably coupled to the handpiece.

9. The small-gauge optical element of claim 8, wherein the optical cable gauge is equal to the gauge of the optical fiber.

10. The small-gauge optical element of claim 8, wherein the optical cable comprises a plurality of optical fibers.

11. The small-gauge optical element of claim 8, wherein the first and second optical connectors are SMA optical fiber connectors.

12. The small-gauge optical element of claim 5, wherein the optical fiber is operably coupled to the handpiece to enable linear displacement of the optical fiber and the small-gauge optical element within the cannula.

13. The small-gauge optical element of claim 12, wherein the handpiece further comprises a means for adjusting the linear displacement.

14. The small-gauge optical element of claim 13, wherein the adjusting means comprises a push/pull mechanism.

15. The small-gauge optical element of claim 13, wherein a distal end of the small-gauge optical element is co-incident with an open aperture of the cannula.

16. The small-gauge optical element of claim 15, wherein adjusting the linear displacement adjusts the small-gauge optical element position relative to the open aperture by an amount corresponding to the change in linear displacement.

17. The small-gauge optical element of claim 16, wherein the amount of linear displacement of the small-gauge optical element determines an angle of illumination and an amount of illumination from the light beam provided to illuminate the surgical field.

18. The small-gauge optical element of claim 17, wherein the angle of illumination can be varied between about 20 degrees to greater than about 180 degrees.

19. The small-gauge optical element of claim 1, wherein the light beam comprises a beam of spatially and temporally incoherent light having a broad spectral bandwidth.

20. The small-gauge optical element of claim 1, wherein the light source further comprises one or more optical filters to selectively illuminate the surgical field with different colors of light, such as to excite a surgical dye.

21. The small-gauge optical element of claim 1, wherein the light source is a xenon or halogen light source.

22. The small-gauge optical element of claim 1, wherein the polymer matrix is closely index-matched to an environment of the surgical field.

23. The small-gauge optical element of claim 22, wherein the environment is the interior of an eye.

24. The small-gauge optical element of claim 1, wherein the plurality of microbubbles is randomly distributed within the polymer matrix.

25. The small-gauge optical element of claim 1, wherein the plurality of microbubbles each have a diameter of about 1 to 50 microns.

26. The small-gauge optical element of claim 1, wherein the plurality of microbubbles is distributed with a distribution density operable to scatter and transmit the light beam in an isotropic manner.

27. The small-gauge optical element of claim 1, wherein the polymer matrix is manufactured from one of the group of clear optical grade epoxy resin, uv curable optical adhesive, uv curable resin, glass, optically transparent ceramic material and transparent silicone rubber.

28. The small-gauge optical element of claim 1, wherein the microbubbles comprise one of the group of thermally expanding plastic microspheres and gas-filled spheroidal microbubbles.

29. The small-gauge optical element of claim 1, wherein the small-gauge optical element is about 2 millimeters long.

30. A small-gauge, wide-angle illuminator, comprising:
a handpiece, optically coupled to receive a light beam from a light source;
an optical fiber, operably coupled to the handpiece, wherein the optical fiber receives the light beam from the light source;
an optical element, optically coupled to a distal end of the optical fiber, for receiving the light beam and scattering the light beam to illuminate a surgical field, wherein the optical element comprises:
a polymer matrix; and
a plurality of microbubbles displaced within the polymer matrix; and
a cannula, operably coupled to the handpiece, for housing and directing the optical fiber and the optical element.

31. The small-gauge, wide-angle illuminator of claim 30, wherein the optical element is a small-gauge optical element comprising circular or semi-ellipsoidal incident surfaces.

32. The small-gauge, wide-angle illuminator of claim 30, wherein the optical element is sized between 18 and 30 gauge.

33. The small-gauge, wide-angle illuminator of claim 30, wherein the optical element, the cannula and the handpiece are fabricated from biocompatible materials.

34. The small-gauge, wide-angle illuminator of claim 30, wherein the optical fiber is optically coupled at the distal end to the optical element and at another end to an optical cable, wherein the optical cable is operably coupled to the light source to transmit the light beam to the optical fiber, and wherein the optical cable comprises a first optical connector operably coupled to the light source and a second optical connector operably coupled to the handpiece.

35. The small-gauge, wide-angle illuminator of claim 34, wherein the optical cable gauge is equal to the gauge of the optical fiber.

36. The small-gauge, wide-angle illuminator of claim 34, wherein the optical cable comprises a plurality of optical fibers.

37. The small-gauge, wide-angle illuminator of claim 34, wherein the first and second optical connectors are SMA optical fiber connectors.

38. The small-gauge, wide-angle illuminator of claim 30, wherein the optical fiber gauge and the optical element gauge are equal.

39. The small-gauge, wide-angle illuminator of claim 30, wherein the optical fiber is operably coupled to the handpiece to enable linear displacement of the optical fiber and the optical element within the cannula.

40. The small-gauge, wide-angle illuminator of claim 39, wherein the handpiece further comprises a means for adjusting the linear displacement.

41. The small-gauge, wide-angle illuminator of claim 40, wherein the adjusting means comprises a push/pull mechanism.

42. The small-gauge, wide-angle illuminator of claim 40, wherein a distal end of the optical element is co-incident with an open aperture of the cannula.

43. The small-gauge, wide-angle illuminator of claim 42, wherein adjusting the linear displacement adjusts the optical element position relative to the open aperture by an amount corresponding to the change in linear displacement.

44. The small-gauge, wide-angle illuminator of claim 43, wherein the amount of linear displacement of the optical element determines an angle of illumination and an amount of illumination from the light beam provided to illuminate the surgical field.

45. The small-gauge, wide-angle illuminator of claim 44, wherein the angle of illumination can be varied between about 20 degrees to greater than about 180 degrees.

46. The small-gauge, wide-angle illuminator of claim 30, wherein the light beam comprises a beam of spatially and temporally incoherent light having a broad spectral bandwidth.

47. The small-gauge, wide-angle illuminator of claim 30, wherein the light source is a xenon or halogen light source.

48. The small-gauge, wide-angle illuminator of claim 30, wherein the light source further comprises one or more optical filters to selectively illuminate the surgical field with different colors of light, such as to excite a surgical dye.

49. The small-gauge, wide-angle illuminator of claim 30, wherein the polymer matrix is closely index-matched to an environment of the surgical field.

50. The small-gauge, wide-angle illuminator of claim 30, wherein the environment is the interior of an eye.

51. The small-gauge, wide-angle illuminator of claim 30, wherein the plurality of microbubbles is randomly distributed within the polymer matrix.

52. The small-gauge, wide-angle illuminator of claim 30, wherein the plurality of microbubbles each have a diameter of about 1 to 50 microns.

53. The small-gauge, wide-angle illuminator of claim 30, wherein the plurality of microbubbles is distributed with a distribution density operable to scatter and transmit the light beam in an isotropic manner.

54. The small-gauge, wide-angle illuminator of claim 30, wherein the polymer matrix is manufactured from one of the group of clear optical grade epoxy resin, uv curable optical adhesive, uv curable resin, glass, optically transparent ceramic material and transparent silicone rubber.

55. The small-gauge, wide-angle illuminator of claim 30, wherein the microbubbles comprise one of the group of thermally expanding plastic microspheres and gas-filled spheroidal microbubbles.

56. The small-gauge, wide-angle illuminator of claim 30, wherein the optical element is about 2 millimeters long.

57. A small-gauge, wide-angle illumination surgical system comprising:
a light source for providing a light beam;
an optical cable, optically coupled to the light source for receiving and transmitting the light beam;
a handpiece, operably coupled to the optical cable to receive the light beam;
an optical fiber, operably coupled to the handpiece, wherein the optical fiber is optically coupled to the optical cable to receive and transmit the light beam;
an optical element, optically coupled to a distal end of the optical fiber, for receiving the light beam and scattering the light beam to illuminate a surgical field, wherein the optical element comprises:
a polymer matrix; and
a plurality of microbubbles displaced within the polymer matrix; and
a cannula, operably coupled to the handpiece, for housing and directing the optical fiber and the optical element.

58. The small-gauge, wide-angle illumination surgical system of claim 57, wherein the optical element is a small-gauge optical element comprising circular or semi-ellipsoidal incident surfaces.

59. The small-gauge, wide-angle illumination surgical system of claim 57, wherein the optical element is sized between 18 and 30 gauge.

60. The small-gauge, wide-angle illumination surgical system of claim 57, wherein the optical element, the cannula and the handpiece are fabricated from biocompatible materials.

61. The small-gauge, wide-angle illumination surgical system of claim 57, wherein the optical fiber is an integral part of the optical cable.

62. The small-gauge, wide-angle illumination surgical system of claim 57, wherein the optical cable comprises a first optical connector operably coupled to the light source and a second optical connector operably coupled to the handpiece.

63. The small-gauge, wide-angle illumination surgical system of claim 62, wherein the first and second optical connectors are SMA optical fiber connectors.

64. The small-gauge, wide-angle illumination surgical system of claim 57, wherein the optical cable gauge is equal to the gauge of the optical fiber.

65. The small-gauge, wide-angle illumination surgical system of claim 57, wherein the optical cable comprises a plurality of optical fibers.

66. The small-gauge, wide-angle illumination surgical system of claim 57, wherein the optical fiber gauge and the optical element gauge are equal.

67. The small-gauge, wide-angle illumination surgical system of claim 57, wherein the optical fiber is operably coupled to the handpiece to enable linear displacement of the optical fiber and the optical element within the cannula.

68. The small-gauge, wide-angle illumination surgical system of claim 67, wherein the handpiece further comprises a means for adjusting the linear displacement.

69. The small-gauge, wide-angle illumination surgical system of claim 68, wherein the adjusting means comprises a push/pull mechanism.

70. The small-gauge, wide-angle illumination surgical system of claim 68, wherein a distal end of the optical element is co-incident with an open aperture of the cannula.

71. The small-gauge, wide-angle illumination surgical system of claim 70, wherein adjusting the linear displacement adjusts the small-gauge optical element position relative to the open aperture by an amount corresponding to the change in linear displacement.

72. The small-gauge, wide-angle illumination surgical system of claim 71, wherein the amount of linear displacement of the optical element determines an angle of illumination and an amount of illumination from the light beam provided to illuminate the surgical field.

73. The small-gauge, wide-angle illumination surgical system of claim 72, wherein the angle of illumination can be varied between about 20 degrees to greater than about 180 degrees.

74. The small-gauge, wide-angle illumination surgical system of claim 57, wherein the light beam comprises a beam of spatially and temporally incoherent light having a broad spectral bandwidth.

75. The small-gauge, wide-angle illumination surgical system of claim 57, wherein the light source further comprises one or more optical filters to selectively illuminate the surgical field with different colors of light, such as to excite a surgical dye.

76. The small-gauge, wide-angle illumination surgical system of claim 57, wherein the light source is a xenon or a halogen light source.

77. The small-gauge, wide-angle illumination surgical system of claim 57, wherein the polymer matrix is index-matched to an environment of the surgical field.

78. The small-gauge, wide-angle illumination surgical system of claim 57, wherein the environment is the interior of an eye.

79. The small-gauge, wide-angle illumination surgical system of claim 57, wherein the plurality of microbubbles is randomly distributed within the polymer matrix.

80. The small-gauge, wide-angle illumination surgical system of claim 57, wherein the plurality of microbubbles each have a diameter of about 1 to 50 microns.

81. The small-gauge, wide-angle illumination surgical system of claim 57, wherein the plurality of microbubbles is distributed with a distribution density operable to scatter and transmit the light beam in an isotropic manner.

82. The small-gauge, wide-angle illumination surgical system of claim 57, wherein the polymer matrix is manufactured from one of the group of clear optical grade epoxy resin, uv curable optical adhesive, uv curable resin, glass, optically transparent ceramic material and transparent silicone rubber.

83. The small-gauge, wide-angle illumination surgical system of claim 57, wherein the microbubbles comprise one of the group of thermally expanding plastic microspheres and gas-filled spheroidal microbubbles.

84. The small-gauge, wide-angle illumination surgical system of claim 57, wherein the optical element is about 2 millimeters long.
